(19) United States
(12) Patent Application Publication
(10) Pub. No.: US 2021/0087504 A1
(43) Pub. Date: Mar. 25, 2021

(54) A CLEANING PRODUCT

(71) Applicant: Bunlue KONGCHAI, Pathum Thani (TH)

(72) Inventor: Bunlue KONGCHAI, Pathum Thani (TH)

(21) Appl. No.: 16/635,258

(22) PCT Filed: Dec. 7, 2017
(86) PCT No.: PCT/TH2017/000085

§ 371 Date: Jan. 30, 2020

(30) Foreign Application Priority Data
Jul. 31, 2017 (TH) 1701004288

(51) Int. Cl. C11D 17/04; A47L 13/17; A61K 8/02; A61K 8/37; A61K 8/44; A61K 8/9789; A61Q 19/10; C11D 11/00; C11D 1/94; C11D 3/00; C11D 3/48

(52) U.S. Cl. CPC C11D 17/049; A47L 13/17; A61K 8/0208; A61K 8/37; A61K 8/44; A61K 8/9789; A61K 2800/52; C11D 11/0058; C11D 1/94; C11D 3/0026; C11D 3/48; A61K 2800/10; A61Q 19/10

(57) ABSTRACT

The present invention relates to a cleaning product comprising a first substrate containing cleaning composition for using with a surface, and a second substrate containing a washing composition for removing the cleaning composition from the surface. According to the present invention, the cleaning composition comprises water in an amount of 70-97% by weight and a surfactant in an amount of 5-20% by weight, and the washing composition comprises water in an amount of 70-97% by weight and an anti-foaming agent in an amount of 0.5-10% by weight.

### FIELD OF THE INVENTION

This invention is in a chemical field related to a cleaning product.

### BACKGROUND OF THE INVENTION

The cleaning of human or animal's body or surface of any objects is normally performed by wetting the surface that requires cleaning with water, washing the surface that requires cleaning with a cleaning product such as soap, shower cream or cleaning agent, and rinsing off the cleaning product with water thereafter.

However, the typical cleaning method mentioned above requires water to wet the surface and rinse off the cleaning product, thus wasting water resource. Moreover, this may be inappropriate or impossible in some cases, for example, when having an illness which causes one to be unable to shower, when staying in a place where there is no appropriate washing area or clean water, or in a case when one wants to clean only some body parts in a short amount of time and does not want to wet other body parts.

Therefore, there have been efforts in developing cleaning products which can be used for cleaning without having to use water to wet and clean a surface. Examples of the prior arts related to this field are as follows:

European patent publication no. 1676512 A2 discloses a skin treatment article comprising a first structure having a fibrous layer and a cleansing formulation comprising foaming surfactants, and a second structure having a fibrous layer and a conditioning formulation, said first and second structures are contained separately with an impervious layer between the two structures. However, the skin treatment article according to the invention is a dry article which, when using, the user must wet the article with water first to produce foam, so it can be used to clean the skin.

European patent publication no. 1757261 A2 discloses a wet cleaning product comprising a porous sheet which is impregnated with active ingredients by spraying, soaking or immersing. The active ingredients according to the invention comprise main components which are 63-98% by weight of water and 0.6-10% by weight of a surfactant. However, the invention does not disclose the use of an anti-foaming agent as a component in the active ingredients. Therefore, when the user uses the product for cleaning purpose, there is a chance that the foam will remain on the skin, causing irritation and a sticky feeling to the user's skin.

U.S. Pat. No. 5,951,991 discloses a disposable cleaning product that is substantially dry prior to use. Such product comprises a water-insoluble substrate and a conditioning formulation comprising a foaming surfactant in an amount of 4-10% by weight. Similarly, the cleaning product according to the invention is dry. Therefore, the user needs to wet the product with water prior to use, which can be inconvenient. Moreover, the conditioning formulation according to this invention does not comprise an anti-foaming agent; as a result, when the user uses the product for cleaning purpose, there is a chance that the foam will remain on the skin, causing irritation and a sticky feeling to the skin of the user.

### SUMMARY OF THE INVENTION

The present invention relates to a cleaning product comprising a first substrate containing a cleaning composition for using with a surface, and a second substrate containing a washing composition for removing the cleaning composition from the surface. The cleaning composition comprising water in an amount of 70-97% by weight and a surfactant in an amount of 5-20% by weight, and the washing composition comprising water in an amount of 70-97% by weight and an anti-foaming agent in an amount of 0.5-10% by weight.

Preferably, the cleaning composition of the cleaning product according to this invention comprises:

a. water in an amount of 70-97% by weight, preferably 90-95% by weight;

b. a surfactant in an amount of 5-20% by weight, preferably 5-15% by weight;

c. a preservative in an amount of 0.1-2% by weight, and

d. a stabilizer in an amount of 0.1-2% by weight.

More preferably, the washing composition of the cleaning product according to this invention comprises:

a. water in an amount of 70-97% by weight, preferably 90-97% by weight;

b. an anti-foaming agent in an amount of 0.5-10% by weight, preferably 1-5% by weight;

c. a preservative in an amount of 0.1-2% by weight;

d. a stabilizer in an amount of 0.1-2% by weight, and

e. optionally, a surfactant in an amount of 0.1-2% by weight.

An object of the present invention is to develop the cleaning product that is unique and advantageous so that it is convenient to bring along and ready to be used at all times.

The cleaning product can be used for cleaning purpose without having to use water. It also provides a soft and clean feeling when applying to the surface without leaving the stickiness on the surface, due to the use of gentle chemical composition that does not irritate the surface.

### DETAILED DESCRIPTION OF THE INVENTION

The present invention will be described in more detail hereafter.

Use of singular nouns or pronouns when used with “comprising” in claims and/or specification means “one” and will also include “one or more”, “at least one”, and “one or more than one”.

The term “% by weight” according to the present invention which indicates amounts of components, substances or ingredients, unless indicated otherwise, means “% by weight of the cleaning composition” or “% by weight of the washing composition”, depending on the purpose of using the composition.

The term “surface” according to the present invention means any surfaces including, but not limited to, the surface of face, arm, leg, torso, bottom, chest, neck, hand, foot or any surfaces on human or animal's body, and may include the surface of floor or any objects to which the cleaning product will be applied.

Cleaning Product

The cleaning product according to the present invention comprises the first substrate containing the cleaning composition for using with the surface, and the second substrate containing the washing composition for removing the cleaning composition from the surface.

According to this invention, the cleaning product is characterized in that the cleaning composition comprises water in the amount of 70-97% by weight and the surfactant in the amount of 5-20% by weight, and the washing composition comprises water in the amount of 70-97% by weight and the anti-foaming agent in the amount of 0.5-10% by weight.

According to this invention, the cleaning product comprises a first substrate and a second substrate which may be contained together or separately in the cleaning product. Preferably, the first substrate and second substrate are contained separately in the cleaning product.

According to this invention, the cleaning product comprises the first substrate containing the cleaning composition in an amount of 300-700% by weight, based on dry weight of the substrate, and the second substrate containing the washing composition in an amount of 300-700% by weight, based on dry weight of the substrate.

Substrate

The substrate according to this invention is made of a material which can be selected from natural fibers, for example, cotton, cotton wool, synthetic fiber such as polyester, or blended materials obtained from natural fiber and synthetic fiber, for example, the blend material of 40-100% natural fiber and 0-60% synthetic fiber. The substrate according to the present invention may be made of woven fabric, nonwoven fabric or paper.

Examples of preferred materials which can be selected as a substrate according to this invention include, but are not limited to, cotton, lint, synthetic fabric or cotton-polyester fabric.

According to this invention, the substrate may be smooth, non-patterned, embossing-patterned, or porous.

According to this invention, the substrate may have a length of from 5-50 cm, preferably from 20-40 cm, more preferably from 25-35 cm, and a width of from 5-50 cm, preferably from 10-30 cm, more preferably from 15-25 cm.

According to this invention, the substrate may have a weight in g/m² ranging from 60-120 g/m², preferably from 80-100 g/m².

Cleaning Composition

The cleaning composition according to this invention comprises water in the amount of 70-97% by weight, the surfactant in the amount of 5-20% by weight, the preservative in the amount of 0.1-2% by weight, and the stabilizer in the amount of 0.1-2% by weight.

Preferably, the cleaning composition according to this invention comprises water in the amount of 90-95% by weight, the surfactant in the amount of 5-15% by weight, the preservative in the amount of 0.1-2% by weight, and the stabilizer in the amount of 0.1-2% by weight.

Washing Composition

The washing composition according to this invention comprises water in the amount of 70-97% by weight, the anti-foaming agent in the amount of 0.5-10% by weight, the preservative in the amount of 0.1-2% by weight, the stabilizer in the amount of 0.1-2% by weight, and optionally the surfactant in the amount of 0.1-2% by weight.

Preferably, the washing composition according to this invention comprises water in the amount of 90-97% by weight, the anti-foaming agent in the amount of 1-5% by weight, the preservative in the amount of 0.1-2% by weight, the stabilizer in the amount of 0.1-2% by weight, and optionally the surfactant in the amount of 0.1-2% by weight.

Surfactant

According to this invention, the surfactant for the cleaning composition and the washing composition can be selected from a cationic surfactant, an anionic surfactant, a nonionic surfactant, an amphoteric surfactant or a mixture thereof.

Preferably, the surfactant for the cleaning composition is the mixture of the nonionic surfactant and the amphoteric surfactant.

Preferably, the surfactant for the washing composition is the nonionic surfactant.

According to this invention, the nonionic surfactant can be selected from olive oil PEG-7 ester, PEG-40 hydrogenated castor oil, polyoxyethylene sorbitan monooleate, polyoxyethylene sorbitan monolaurate, soap nut extract or a mixture thereof.

According to this invention, the anionic surfactant can be selected from sodium lauryl sulfate, sodium laurate sulfate, alkylbenzene sulfonate or a mixture thereof.

According to this invention, the amphoteric surfactant can be selected from betaine surfactant, preferably cocamidopropyl betaine.

According to this invention, a preferred surfactant for the cleaning composition is a mixture of cocamidopropyl betaine and PEG-40 hydrogenated castor oil.

Preferably, the surfactant for the cleaning composition is a mixture of cocamidopropyl betaine in an amount of 5-10% by weight and PEG-40 hydrogenated castor oil in an amount of 0.5-1.5% by weight.

According to this invention, a preferred surfactant for the washing composition is PEG-40 hydrogenated castor oil.

Anti-Foaming Agent

The anti-foaming agent according to this invention can be selected from a group consisting of fatty alcohol, polydimethylsiloxane emulsion, calcium carbonate, magnesium carbonate or a mixture thereof.

According to this invention, a preferred anti-foaming agent is polydimethylsiloxane emulsion.

Preservative

The preservative according to this invention can be selected from a group consisting of butylated hydroxytoluene, phenoxyethanol, sodiummetabisulfite, chlorphenesin, Lonicera japonica (honeysuckle) flower extract, Usnea barbata (lichen) extract, Citrus paradisi (grapefruit) extract, Ocimum basilicum extract, benzyl alcohol, ethylhexylglycerin, tocopherol, benzoic acid, dehydroacetic acid, sodium benzoate, potassium sorbate or a mixture thereof.

The exemplary mixtures of the preservative which can be used according to the present invention include, but are not limited to, a mixture of benzyl alcohol, ethylhexylglycerin and tocopherol, a mixture of benzyl alcohol, benzoic acid and dehydroacetic acid or a mixture of sodium benzoate and potassium sorbate.

According to this invention, a preferred preservative is phenoxyethanol.

Stabilizer

The stabilizer according to this invention can be selected from a group consisting of ethylenediaminetetraacetic acid (EDTA), metal alkaline salt and derivatives of ethylenediaminetetraacetic acid such as disodium salt of ethylenediaminetetraacetic acid (disodium EDTA), tetrasodium salt of ethylenediaminetetraacetic acid (tetra sodium EDTA) or a mixture thereof.

According to this invention, a preferred stabilizer is disodium salt of ethylenediaminetetraacetic acid.

Additive

The cleaning composition and washing composition according to this invention may further comprise as least one additive which can be selected from an anti-bacterial agent, a moisturizer, an emollient, a skin conditioning agent, an antioxidant, a fragrance, a whitening agent, an anti-pollution agent, a smoothing agent, a protective agent, a sunscreen agent or a mixture thereof.

According to this invention, the anti-bacterial agent can be selected from parachlorometacresol, pine oil, ethylhexylglycerin, chlorhexidine, povidone iodine, hydrogen peroxide or a mixture thereof.

According to this invention, a preferred anti-bacterial agent is ethylhexylglycerin.

According to this invention, the moisturizer, emollient and/or skin conditioning agent can be selected from tocopheryl acetate, Aloe barbadensis leaf extract, Cucumis sativus fruit extract or a mixture thereof.

According to this invention, the antioxidant can be selected from tocopheryl acetate, Aloe barbadensis leaf extract, Cucumis sativus fruit extract or a mixture thereof.

According to this invention, the fragrance which can be used is, for example, menthol.

### Examples

The present invention will now be described in more detail by way of examples. However, these examples should not be considered limiting the scope of the present invention.

1. Composition

1.1 Cleaning Composition

The Effect of the Amounts of the Ingredients of the Cleaning Composition on the Foaming Property and Cleaning Efficiency

The exemplary cleaning compositions according to this invention can be prepared by heating a mixture of water and the stabilizer until the temperature is in a range of 30−80° C. After that, lower the temperature of the mixture to be in a range of 20−50° C. Then, add the other ingredients and mix well to obtain the cleaning compositions. The obtained cleaning compositions are tested for the foaming property and cleaning efficiency by applying to the voluntary users. The samples of the cleaning compositions are shown in Table 1.

Table 1 shows the ingredients of the samples of the cleaning compositions.

| | Amount (% by weight) | | | |
|---|---|---|---|---|
| Ingredients | Sample 1 | Sample 2 | Sample 3 | Sample 4 |
| Water | 92.3 | 90.3 | 89.3 | 87.3 |
| Disodium EDTA | 0.50 | 0.50 | 0.50 | 0.50 |
| Tocopheryl acetate | 0.01 | 0.01 | 0.01 | 0.01 |
| PEG-40 hydrogenated | 1.00 | 1.00 | 1.00 | 1.00 |
| castor oil | | | | |
| Phenoxyethanol | 0.50 | 0.50 | 0.50 | 0.50 |
| Fragrance | 0.10 | 0.10 | 0.10 | 0.10 |
| Anti-bacterial agent | 0.50 | 0.50 | 0.50 | 0.50 |
| Menthol | 0.10 | 0.10 | 0.10 | 0.10 |
| Cocamidopropyl betaine | 5.00 | 7.00 | 8.00 | 10.00 |

When applying the samples 1-4 to the users, the test results show that the samples 1-4 provide satisfactory large volume of foam and are capable of cleaning off the dirt efficiently. The cleaning compositions containing the amphoteric surfactant that is cocamidopropyl betaine in the amount of 5, 7 and 8% by weight (samples 1-3, respectively) provide foam that is more delicate than that of the cleaning composition containing the amphoteric surfactant in the amount of 10% by weight. Thus, it can be concluded from the test results that the surfactant which is the mixture of the amphoteric surfactant (i.e., cocamidopropyl betaine in the samples) and the nonionic surfactant (i.e., PEG-40 hydrogenated castor oil in the samples) is suitable for using in the cleaning composition according to the present invention.

Further, the test results show that the cleaning compositions with the amounts of the surfactant in the determined range according to the present invention provide preferred foaming property and cleaning efficiency as desired.

1.2 Washing Composition

The Effect of the Amounts of the Ingredients of the Washing Composition on the Efficiency in Removing the Cleaning Composition and Feeling Satisfaction

The exemplary washing compositions according to this invention can be prepared by heating a mixture of water and the stabilizer until the temperature is in a range of 30-80° C. After that, lower the temperature of the mixture to be in a range of 20-50° C. Then, add the other ingredients and mix well to obtain the washing compositions. The obtained washing compositions are tested for the efficiency in removing foam produced by the cleaning compositions and the sensory satisfaction by applying to the voluntary users. The samples of the washing compositions are shown in Table 2.

Table 2 shows the ingredients of the samples of the washing compositions.

| | Amount (% by weight) | | | |
|---|---|---|---|---|
| Ingredients | Sample 5 | Sample 6 | Sample 7 | Sample 8 |
| Water | 96.8 | 96.3 | 95.8 | 95.3 |
| Disodium EDTA | 0.50 | 0.50 | 0.50 | 0.50 |
| Aloe barbadensis leaf | 0.008 | 0.008 | 0.008 | 0.008 |
| extract | | | | |
| Cucumis sativus fruit | 0.01 | 0.01 | 0.01 | 0.01 |
| extract | | | | |
| PEG-40 hydrogenated | 0.98 | 0.98 | 0.98 | 0.98 |
| castor oil | | | | |
| Phenoxyethanol | 0.50 | 0.50 | 0.50 | 0.50 |
| Tocopheryl acetate | 0.01 | 0.01 | 0.01 | 0.01 |
| Anti-bacterial agent | 0.50 | 0.50 | 0.50 | 0.50 |
| Fragrance | 0.05 | 0.05 | 0.05 | 0.05 |
| Menthol | 0.10 | 0.10 | 0.10 | 0.10 |
| Polydimethylsiloxane | 0.50 | 1.00 | 1.50 | 2.00 |
| emulsion | | | | |

When applying the samples 5-8 to the users, the test results show that the samples 5-8 provide effective results in terms of cleaning composition removal (foam removal). The sample 7 which contains the anti-foaming agent that is polydimethylsiloxane emulsion in the amount of 1.5% by weight provides the most satisfying result in terms of cleaning composition removal as it does not leave a white stain nor give the users a sticky feeling after using it. Although the samples 5 and 6 do not give a sticky feeling after using, a white stain was found remaining on the tested surface. After using, it was found that the sample 8 can remove the cleaning composition effectively and does not leave a white stain, but it leaves a slightly sticky feeling on the tested surface.

2. Substrate

2.1 the Effect of the Sizes and Weights of the Substrates on the Foaming Property

The exemplary first substrates comprising the cleaning composition are tested for the foaming property. The first substrates for testing are made of smooth, non-patterned 100% cotton which are varied in size and weight in g/m², as shown in Table 3.

Table 3 shows the sizes and weights of the samples of the substrates and the foaming property test results.

| | | | Test results | |
|---|---|---|---|---|
| | | | | Appearance |
| | Size | Weight | | of substrate |
| Sample | (cm × cm) | (g/m²) | Foaming property | while using |
| 1 | 36 × 20 | 100 | good foaming property, | no folding |
| | | | delicate foam | |
| 2 | 36 × 20 | 85 | good foaming property, | no folding |
| | | | delicate foam | |
| 3 | 36 × 20 | 80 | good foaming property | slight folding |
| 4 | 36 × 20 | 60 | good foaming property | more folding |
| 5 | 36 × 20 | 40 | good foaming property | more folding |
| 6 | 30 × 20 | 100 | good foaming property, | no folding |
| | | | delicate foam | |
| 7 | 30 × 20 | 85 | good foaming property, | no folding |
| | | | delicate foam | |
| 8 | 30 × 20 | 80 | good foaming property | slight folding |
| 9 | 30 × 20 | 60 | good foaming property | more folding |
| 10 | 30 × 20 | 40 | good foaming property | more folding |

The test results show that the preferred substrate weights which result in good foaming property and convenient usage (i.e., the substrates slightly folded or did not fold while applying to the surface or while rubbing the surface) are from 80-100 g/m².

2.2 the Effect of the Sizes and Weights of the Substrates on the Efficiency in Removing the Cleaning Composition

The exemplary second substrates comprising the washing composition are tested for the efficiency in removing foam produced by the cleaning composition. The second substrates for testing are made of 100% cotton with small embossing patterns. The substrates are varied in sizes and weights in g/m², as shown in Table 4.

Table 4 shows the sizes and weights of the samples of the substrates and the test results on the cleaning composition removal efficiency.

| | | | Test results | |
|---|---|---|---|---|
| | | | | Contact |
| | Size | Weight | | feeling while |
| Sample | (cm × cm) | (g/m²) | Foam removal | using substrate |
| 1 | 36 × 20 | 100 | foam is completely | soft |
| | | | removed, no white stain | |
| | | | remains | |
| 2 | 36 × 20 | 85 | foam is completely | less soft |
| | | | removed, no white stain | |
| | | | remains | |
| 3 | 36 × 20 | 75 | foam is partially | — |
| | | | removed, white stain | |
| | | | remains | |
| 4 | 36 × 20 | 60 | foam is partially | — |
| | | | removed, white stain | |
| | | | remains | |
| 5 | 30 × 20 | 100 | foam is completely | soft |
| | | | removed, no white stain | |
| | | | remains | |
| 6 | 30 × 20 | 85 | foam is completely | less soft |
| | | | removed, no white stain | |
| | | | remains | |
| 7 | 30 × 20 | 75 | foam is partially | — |
| | | | removed, white stain | |
| | | | remains | |
| 8 | 30 × 20 | 60 | foam is partially | — |
| | | | removed, white stain | |
| | | | remains | |

The test results show that the appropriate substrate weights which result in an effective cleaning composition removal and give a gentle feeling or a mild contact against the surface are from 85-100 g/m².

2.3 the Effect of the Material Types and Patterns of the Substrates on the Foaming Property

The exemplary first substrates comprising the cleaning composition are tested for the foaming property to compare the types and patterns of the materials used to form the substrates which affect the foaming property. The test results are shown in Table 5.

Table 5 shows the material types and patterns of the substrates and the foaming property test results.

| | | Material | |
|---|---|---|---|
| Sample | Material type | pattern | Foaming property |
| 1 | 100% cotton | smooth, | good foaming property, |
| | | non- | delicate foam |
| | | patterned | |
| 2 | 100% cotton | porous | good foaming property |
| 3 | 100% cotton | embossing- | good foaming property, |
| | | patterned | delicate foam |
| 4 | 70% cotton mixed with | smooth, | poor foaming property |
| | 30% synthetic fiber | non- | |
| | | patterned | |
| 5 | 70% cotton mixed with | porous | poor foaming property |
| | 30% synthetic fiber | | |
| 6 | 70% cotton mixed with | embossing- | poor foaming property |
| | 30% synthetic fiber | patterned | |

The test results show that the substrates made of 100% cotton (samples 1-3) allow the cleaning composition to produce foam more effectively than the substrates made of mixed materials between cotton and synthetic fiber (samples 4-6).

2.4 the Effect of the Material Types and Patterns of the Substrates on the Cleaning Composition Removal Efficiency

The exemplary second substrates comprising the washing composition are tested for the foam removal property to compare the types and patterns of the materials to see how they affect the foam removal and the usability. The test results are shown in Table 6.

Table 6 shows the material types and patterns of the substrates and the test results on the cleaning composition removal efficiency.

| Sample | Material type | Material pattern | Foam removal |
|---|---|---|---|
| 1 | 100% cotton | smooth, | foam is completely removed, |
| | | non-patterned | no white stain remains |
| 2 | 100% cotton | porous | foam is completely removed |
| 3 | 100% cotton | embossing- | foam is completely removed, |
| | | patterned | no white stain remains |

The test results show that the substrates, whether it be smooth, non-patterned, porous or embossing-patterned, provide an efficient cleaning composition removal.

3. Cleaning Product

The Effect of the Amounts of the Compositions in the Substrates on the Foaming Property, Cleaning Efficiency, and Removal of Foam Produced by the Cleaning Composition

The exemplary cleaning products according to this invention can be prepared by impregnating, coating or spraying the exemplary first and second substrates with the cleaning composition and washing composition, respectively. Then, press the exemplary substrates containing said compositions to remove excess compositions to obtain the substrates with the amount of composition ranging from 100-800% by weight, based on dry weight of the substrate. Then, test the foaming property and foam removal of the first substrate containing the cleaning composition and the second substrate containing the washing composition by applying with voluntary users. The results are shown in Table 7.

Table 7 shows the amounts of the cleaning composition and washing composition in the first substrate and second substrate, respectively, and the test results on the foaming property and the removal of foam produced by the cleaning composition of said substrates.

| | Amount (% by | | | |
|---|---|---|---|---|
| | weight, based on dry | | | |
| | weight of substrate) | | | |
| | Cleaning | Washing | Test result | |
| | com- | com- | Foaming | Foam |
| Sample | position | position | property | removal |
| 1 | 100 | 100 | The amount of the | — |
| | | | composition is too | |
| | | | small, therefore it | |
| | | | does not disperse | |
| | | | throughout the | |
| | | | substrate. There is a | |
| | | | small amount of | |
| | | | foam. The substrate | |
| | | | is difficult to use as | |
| | | | it is not slippery | |
| | | | when rubbing onto | |
| | | | the surface. | |
| 2 | 200 | 200 | The amount of the | The amount of the |
| | | | composition is too | composition is too |
| | | | small, therefore it | small, therefore the |
| | | | does not disperse | composition does |
| | | | throughout the | not disperse |
| | | | substrate. There is | throughout the |
| | | | an average amount | substrate. The |
| | | | of foam. The | foam is removed |
| | | | substrate is easy to | partially. |
| | | | use and does not | Problems occur |
| | | | cause any problems | when rubbing onto |
| | | | when rubbing onto | the surface. |
| | | | the surface. | |
| 3 | 300 | 300 | The amount of the | The amount of the |
| | | | composition is | composition is |
| | | | appropriate, | appropriate, |
| | | | therefore the | therefore the |
| | | | composition | composition |
| | | | disperses | disperses |
| | | | thoroughly | thoroughly in |
| | | | in the substrate. | the substrate. The |
| | | | There is a large | foam is removed |
| | | | amount of foam. | effectively. |
| | | | No problem occurs | No problem occurs |
| | | | when rubbing the | when rubbing the |
| | | | substrate onto the | substrate onto the |
| | | | surface. | surface. |
| 4 | 500 | 500 | The amount of the | The amount of the |
| | | | composition is | composition is |
| | | | appropriate, | appropriate, |
| | | | therefore the | therefore the |
| | | | composition | composition |
| | | | disperses | disperses |
| | | | thoroughly | thoroughly in |
| | | | in the substrate. | the substrate. The |
| | | | There is a large | foam is removed |
| | | | amount of foam. | effectively. |
| | | | No problem occurs | No problem occurs |
| | | | when rubbing the | when rubbing the |
| | | | substrate onto the | substrate onto the |
| | | | surface. | surface. |
| 5 | 700 | 700 | The amount of the | The amount of the |
| | | | composition is | composition is |
| | | | appropriate, | appropriate, |
| | | | therefore the | therefore the |
| | | | composition | composition |
| | | | disperses | disperses |
| | | | thoroughly | thoroughly in |
| | | | in the substrate. | the substrate. The |
| | | | There is a large | foam is removed |
| | | | amount of foam. | effectively. |
| | | | No problem occurs | No problem occurs |
| | | | when rubbing the | when rubbing the |
| | | | substrate onto the | substrate onto the |
| | | | surface. | surface. |
| 6 | 800 | 800 | The amount of the | The amount of the |
| | | | composition is too | composition is too |
| | | | large, making it | large, making it |
| | | | inconvenient to use | inconvenient to use |
| | | | the substrate as the | the substrate as the |
| | | | composition spills | composition spills |
| | | | out while using. | out. The foam is |
| | | | There is a large | removed |
| | | | volume of foam. | effectively. |

The test results show that the appropriate amounts of the cleaning composition and washing composition in the first substrate and second substrate, respectively, are in a range of 300-700% by weight, based on dry weight of the substrate, as the amounts in such range allow the compositions to disperse thoroughly, provide good foaming property, and remove the foam effectively while it is still easy and convenient to use the substrate and the composition does not spill out while using.

With regard to the details described above, the cleaning product which comprises the first substrate containing the cleaning composition and the second substrate containing the washing composition according to the present invention has the substrate sizes and weights, ingredients, and the amounts of ingredients of the cleaning and washing compositions in an appropriate range. This provides various advantages when using the cleaning product of the present invention for cleaning purpose as described above.

However, the cleaning product of this invention is not limited to the substrate, cleaning composition, washing composition, amounts of ingredients of such compositions, procedures, and details described above, but changes and modifications may be made within the concept and scope of this invention.

1. A cleaning product comprising:
a first substrate containing a cleaning composition for using with a surface; and
a second substrate containing a washing composition for removing the cleaning composition from the surface
characterized in that
the cleaning composition comprises water in an amount of 70-97% by weight and a surfactant in an amount of 5-20% by weight, and
the washing composition comprises water in an amount of 70-97% by weight and an anti-foaming agent in an amount of 0.5-10% by weight.

2. The cleaning product according to claim 1, wherein the cleaning composition comprises: a. water in an amount of 70-97% by weight, preferably 90-95% by weight;
b. a surfactant in an amount of 5-20% by weight, preferably 5-15% by weight;
c. a preservative in an amount of 0.1-2% by weight, and
d. a stabilizer in an amount of 0.1-2% by weight.

3. The cleaning product according to claim 1, wherein the washing composition comprises: a. water in an amount of 70-97% by weight, preferably 90-97%) by weight;
b. an anti-foaming agent in an amount of 0.5-10% by weight, preferably 1-5% by weight;
c. a preservative in an amount of 0.1-2% by weight;
d. a stabilizer in an amount of 0.1-2% by weight, and
e. optionally, a surfactant in an amount of 0.1-2% by weight.

4. The cleaning product according to claim 1, wherein the surfactant is selected from a group consisting of a nonionic surfactant being selected from olive oil PEG-7 ester, PEG-40 hydrogenated castor oil, polyoxyethylene sorbitan monooleate, polyoxyethylene sorbitan monolaurate, soap nut extract, a anionic surfactant being selected from sodium lauryl sulfate, sodium laurate sulfate, alkylbenzene sulfonate, amphoteric surfactant which is cocamidopropyl betaine or a mixture thereof, preferably, the surfactant for the cleaning composition is a mixture of the amphoteric surfactant and the nonionic surfactant, and the surfactant for the washing composition is the nonionic surfactant.

5. The cleaning product according to claim 1, wherein the surfactant for the cleaning composition is a mixture of cocamidopropyl betain in an amount of 5-10% by weight and PEG-40 hydrogenated castor oil in an amount of 0.5-1.5% by weight, and the surfactant for the washing composition is PEG-40 hydrogenated castor oil in an amount of 0.5-1.5% by weight.

6. The cleaning product according to claim 1, wherein the anti-foaming agent is selected from a group consisting of fatty alcohol, polydimethylsiloxane, calcium carbonate, magnesium carbonate or a mixture thereof, preferably, polydimethylsiloxane emulsion.

7. The cleaning product according to claim 2, wherein
the preservative is selected from a group consisting of butylated hydroxytoluene, phenoxyethanol, sodium metabisulfite, chlorphenesin, Lonicera japonica (honeysuckle) flower extract, Usnea barbata (lichen) extract, Citrus paradisi (grapefruit) extract, Ocimum basilicum extract, benzyl alcohol, ethylhexylglycerin, tocopherol, benzoic acid, dehydroacetic acid, sodium benzoate, potassium sorbate or a mixture thereof, preferably, phenoxyethanol, and
the stabilizer is selected from a group consisting of ethylenediaminetetraacetic acid (EDTA), alkali metal salt and ethylenediaminetetraacetic acid derivatives or a mixture thereof, preferably, disodium salt of ethylenediaminetetraacetic acid (disodium EDTA).

8. The cleaning product according to claim 1, wherein the cleaning composition and the washing composition further comprise at least one additive being selected from an anti-bacterial agent, a moisturizer, an emollient, a skin conditioning agent, an antioxidant, a fragrance, a whitening agent, an anti-pollution agent, a smoothing agent, a protective agent, a sunscreen agent or a mixture thereof.

9. The cleaning product according to claim 1, wherein each of the first substrate and second substrate has a weight in a range of 80-100 g/m².

10. The cleaning product according to claim 1, wherein the first substrate contains the cleaning composition in an amount of 300-700% by weight, based on dry weight of the substrate, and the second substrate contains the washing composition in an amount of 300-700% by weight, based on dry weight of the substrate.

11. The cleaning product according to claim 2, wherein the surfactant is selected from a group consisting of a nonionic surfactant being selected from olive oil PEG-7 ester, PEG-40 hydrogenated castor oil, polyoxyethylene sorbitan monooleate, polyoxyethylene sorbitan monolaurate, soap nut extract, a anionic surfactant being selected from sodium lauryl sulfate, sodium laurate sulfate, alkylbenzene sulfonate, amphoteric surfactant which is cocamidopropyl betaine or a mixture thereof, preferably, the surfactant for the cleaning composition is a mixture of the amphoteric surfactant and the nonionic surfactant, and the surfactant for the washing composition is the nonionic surfactant.

12. The cleaning product according to claim 3, wherein the surfactant is selected from a group consisting of a nonionic surfactant being selected from olive oil PEG-7 ester, PEG-40 hydrogenated castor oil, polyoxyethylene sorbitan monooleate, polyoxyethylene sorbitan monolaurate, soap nut extract, a anionic surfactant being selected from sodium lauryl sulfate, sodium laurate sulfate, alkylbenzene sulfonate, amphoteric surfactant which is cocamidopropyl betaine or a mixture thereof, preferably, the surfactant for the cleaning composition is a mixture of the amphoteric surfactant and the nonionic surfactant, and the surfactant for the washing composition is the nonionic surfactant.

13. The cleaning product according to claim 3, wherein the anti-foaming agent is selected from a group consisting of fatty alcohol, polydimethylsiloxane, calcium carbonate, magnesium carbonate or a mixture thereof, preferably, polydimethylsiloxane emulsion.

14. The cleaning product according to claim 3, wherein the preservative is selected from a group consisting of butylated hydroxytoluene, phenoxyethanol, sodium metabisulfite, chlorphenesin, Lonicera japonica (honeysuckle) flower extract, Usnea barbata (lichen) extract, Citrus paradisi (grapefruit) extract, Ocimum basilicum extract, benzyl alcohol, ethylhexylglycerin, tocopherol, benzoic acid, dehydroacetic acid, sodium benzoate, potassium sorbate or a mixture thereof, preferably, phenoxyethanol, and
the stabilizer is selected from a group consisting of ethylenediaminetetraacetic acid (EDTA), alkali metal salt and ethylenediaminetetraacetic acid derivatives or a mixture thereof, preferably, disodium salt of ethylenediaminetetraacetic acid (disodium EDTA).
